(19) United States
(12) Patent Application Publication
(10) Pub. No.: US 2024/0240129 A1
(43) Pub. Date: Jul. 18, 2024

(54) PROBE FOR A BIOREACTOR

(71) Applicant: ABER INSTRUMENTS LIMITED, Aberystwyth, Ceredigion (GB)

(72) Inventors: Colin MCELDUFF, Aberystwyth, Ceredigion (GB); Steffan JONES, Aberystwyth, Ceredigion (GB); Paul YEOMANS, Aberystwyth, Ceredigion (GB)

(21) Appl. No.: 18/562,024

(22) PCT Filed: May 19, 2022
(86) PCT No.: PCT/GB2022/051267

§ 371 Date: Nov. 17, 2023

(30) Foreign Application Priority Data
May 19, 2021 (GB) 2107157.6

(51) Int. Cl. C12M 1/34; C12M 1/12

(52) U.S. Cl. CPC C12M 41/00; C12M 37/04

(57) ABSTRACT

This invention relates to a sensing probe (100, 110) for operatively engaging with a port (200) that comprises a channel (230) that communicates between the interior and the exterior of a bioreactor. The probe (100, 110) comprises an elongate body comprising a first portion (122) that is locatable within the port channel (230) and a second portion (124) comprising means for enabling the probe (100, 110) to be secured to the port (200), and a sensing means (140) located on the first body portion (122), wherein the probe (100, 110) is configured to operatively engage with the port (200) such that the first body portion (122) is at least partially located within the port channel (230) and the sensing means (140) is exposed to the interior of the bioreactor.

The present invention relates to a sensing probe and associated assemblies and methods of use.

Capacitance measurement techniques are known for measuring the capacitance (or the specific capacitance or dielectric constant) of liquids and suspensions, such as a biological medium comprising biological cells in ionic aqueous solutions. Monitoring systems incorporating such measurement capability are beneficial for measuring the concentration of live cells in the biological medium.

In the brewing industry, the concentration of live yeast cells can be measured with an on-line capacitance probe. A radio frequency applied from electrodes located on the probe causes ions in the suspending medium (for example wort or green beer) and the cytoplasm of the yeast cells to move towards the two respective oppositely charged electrodes. As the plasma membrane of yeast cells is non-conducting, a build-up of charge occurs in the cells and the cells become polarised and essentially act as tiny capacitors within the medium. Non-viable cells or cells with a damaged membrane do not interfere with the signal as ions can freely move across the membrane and hence a build-up of charge in their cytoplasm cannot occur. Accordingly, the measured capacitance is directly proportional to the amount of viable yeast within a sample over a wide concentration range.

In addition to the brewing industry, such technology can also be utilised for measuring biomass in the field of biotechnology, for example, in cell culture processes.

In certain applications, whether brewing, biotechnological or other fields of industry, it is desirable to use single-use processing equipment. In such applications, single-use bioreactors may be employed, which are configured to be used for only a single process before disposal. A range of single-use bioreactors are commercially available and are known to those skilled in the art. Examples of such bioreactors have been commercialised under the trade names HyPerforma® by Thermo-Fisher, or FlexSafe® by Sartorius.

In addition to the bioreactors employed in such applications being single-use, it may also be desirable for the components used to monitor the biological medium housed within the bioreactor to also be single-use.

Attempts have been made to provide single-use bioreactors with a sensing probe integrally formed within the walls of the bioreactors. The arrangements disclosed in WO 2010/010313 include configurations in which a sensing probe having a disc-shaped body with an annular flange is positioned within an opening in the bioreactor wall with the annular flange being adhered or welded to the bioreactor wall.

While arrangements of this type do provide a straightforward configuration permitting the monitoring of the biological medium housed within the bioreactor, there are a number of drawbacks associated with their use. In particular, the sensing probe does not project significantly into the bioreactor meaning that, in order for reliable biomass measurements to be taken, the positioning of the probe and the orientation of the bioreactor must be such that the electrodes of the biomass sensing probe are in constant contact with the medium within the bioreactor, which can be problematic depending on the volume of the biomass within the bioreactor or the arrangement or location of the bioreactor. In addition, as the sensing probes are integrally formed with the bioreactor, the user of the bioreactor does not have any choice regarding which sensing probe is used and the entire bioreactor must be disposed of in the event that any of the sensing probes are found to be defective prior to use of the bioreactor. In addition, users wanting to access this sensing technology are restricted to bioreactors produced by manufacturers who offer this specific attachment method, but which may not be compatible with the users' other infrastructure. This bespoke attachment method requires each bioreactor manufacturer to develop their own process to efficaciously secure the sensor due to the different materials and structures used in single-use reactor assemblies, which results in additional time, effort and cost to integrate the sensor.

The system disclosed in WO2019/116043 comprises a sensing probe and a bioreactor that comprises a port that is configured to receive and form a watertight seal with the probe. This arrangement permits a separate sensing probe to be engaged with a proprietary port of a bioreactor in order to monitor the properties of the biological medium housed within the bioreactor.

It is the aim of this invention to provide a sensing probe, such as a probe for taking measurements from a biological medium housed within a bioreactor used in the brewing or biotechnological industries, which overcomes or substantially mitigates the above mentioned and/or other problems associated with the prior art.

Thus, according to a first aspect of the invention, there is provided a sensing probe for operatively engaging with a port that comprises a channel that communicates between the interior and the exterior of a bioreactor, the probe comprising;
an elongate body comprising;
   a first body portion that is locatable within the port channel; and,
   a second body portion comprising means for enabling the probe to be secured to the port; and,
a sensing means located on the first body portion,
   wherein the probe is configured to operatively engage with the port such that the first body portion is at least partially located within the port channel and the sensing means is exposed to the interior of the bioreactor.

The probe according to the first aspect of the invention is capable of operatively engaging with and being secured to a standard port that is conventionally located on bioreactors and, unlike the system disclosed in WO2019/116043, does not require a proprietary port or any other special adaption to the bioreactor in order to function. In addition, although the dimensions and configuration of the probe body are at least partially determined by the dimensions of the port with which the probe is intended to engage, as these ports exist in a range of standard dimensions and configurations, the probe may also be provided in a matching range of standard dimensions and configurations.

The probe according to the first aspect of the invention further permits the sensing probe and bioreactor to be provided as separate components and hence gives the user of the bioreactor a choice of which probes to us. In addition, in the event that the user finds that the sensing probe is defective upon carrying out pre-use validation of the bioreactor, it is only necessary to replace the defective probe, unlike the arrangement disclosed in WO 2010/010313 in which the entire bioreactor must be replaced.

The port with which the probe of this invention may operatively engage is a port of the type that is conventionally located on bioreactors, which comprise a channel that communicates between the interior and the exterior of a bioreactor. The port conventionally permits the transfer of fluids between the interior and the exterior of the bioreactor and may be integral part of the bioreactor wall.

The sensing probe may be for operatively engaging with a port in which the channel is substantially straight and of substantially constant cross-section and cross-sectional area. The longitudinal axis of the port channel is typically substantially perpendicular to the plane of the wall of the bioreactor. Although the probe according to this invention can be configured to operatively engage with a port comprising a channel having substantially any cross-sectional shape, the port channel typically has a circular cross-section.

The port with which the probe of this invention may operatively engage may comprise a collar, such as a cylindrical collar, that projects from the exterior of the wall and that defines the channel of the port.

The channel may have a diameter or maximum cross-sectional dimension of about 3/32″ (2.38 mm) to about 1″ (25.4 mm), or more specifically ⅛″ (3.18 mm) to about 1″ (25.4 mm), and preferably about ¾″ (19.05 mm). In particular, the channel may have a diameter or maximum cross-sectional dimension of 3/32″ (2.38 mm), ⅛″ (3.18 mm), 5/32″ (3.97 mm), 3/16″ (4.76 mm), ¼″ (6.35 mm), 5/16″ (7.94 mm), ⅜″ (9.53 mm), ½″ (12.7 mm), ⅝″ (15.88 mm), ¾″ (19.05 mm), 1″ (25.4 mm) or any other diameter or maximum cross-sectional dimension in which ports are available.

The length of the channel may range from about 10 mm, about 20 mm, about 50 mm, about 70 mm, about 100 mm, to about 150 mm, about 200 mm, about 250 mm, about 300 mm, about 350 mm, about 400 mm, about 450 mm, about 500 mm, about 600 mm or about 700 mm. The channel may also have a length that is greater than about 700 mm.

The sensing probe may be for operatively engaging with a port that further comprises one or more projections, for example in order to facilitate the formation of a watertight seal with conventional tubes or other components that are conventionally engaged with the bioreactor port. In particular, ports comprising a collar that projects from the exterior of the wall and that defines the channel of the port may further comprise one or more projections on the exterior surface of the collar. The one or more projections may be annular and extend around the entire circumference of the exterior wall of the collar, and in particular may comprise one or more barbs that comprise a shoulder on the side of the barb that is distal from the exterior end of the collar. This type of port is conventionally referred to as a barbed port and are available from a range of manufacturers including Eldon James Corporation, Value Plastics Inc, Nordson Corporation and products sold under the trade mark Masterflex®.

When the sensing probe is operatively engaged with the port, the sensing means, typically along with at least a portion of the probe body, is exposed to the interior of the bioreactor and is able to obtain measurements from within the bioreactor, such as from a biological medium contained within the bioreactor.

The probe may be configured to operatively engage with the port such that the sensing means is at least partly located in the interior of the bioreactor (ie the sensing means is not entirely located in the port channel). The sensing means being at least partly located in the interior of the bioreactor may improve the exposure of the sensing means to a biological medium contained within the bioreactor and hence improve the ability of the sensing means to obtain measurements from the biological medium.

The first body portion is configured to be locatable within the port channel. In particular, the first body portion may have a diameter or cross-sectional profile that is the same as or smaller than the diameter or cross-sectional profile of the port channel. In particular, the diameter or cross-sectional profile of the first body portion may be the same as or only marginally smaller than the diameter or cross-sectional profile of the port channel such that first body portion of the sensing probe fits tightly within the port channel and hence is held in a stable position by the port channel when it is operatively engaged with the port.

The first body portion may have any cross-sectional shape that can be located within the port channel. In particular, the first body portion may be circular, square, diamond-shaped, rectangular, hexagonal, or another polygonal shape in cross-section. However, the cross-sectional shape of the first body portion is typically the same or substantially the same as the cross-sectional shape of the port channel. Accordingly, as conventional bioreactor ports typically comprise channels that are circular in cross-section, the first body portion may also be circular in cross-section.

The first body portion may have a diameter or maximum cross-sectional dimension that is the same as or smaller than the diameter or maximum cross-sectional dimension of the port channel. In particular, the first body portion of the biomass sensing probe may have a diameter or maximum cross-sectional dimension of between about 3/32″ (2.38 mm) to about 1″ (25.4 mm), or more specifically ⅛″ (3.18 mm) to about 1″ (25.4 mm), and preferably about ¾″ (19.05 mm). In particular, the first body portion of the biomass sensing probe may have a diameter or maximum cross-sectional dimension of, or marginally less than, 3/32″ (2.38 mm), ⅛″ (3.18 mm), 5/32″ (3.97 mm), 3/16″ (4.76 mm), ¼″ (6.35 mm), 5/16″ (7.94 mm), ⅜″ (9.53 mm), ½″ (12.7 mm), ⅝″ (15.88 mm), ¾″ (19.05 mm), 1″ (25.4 mm).

In embodiments in which the first body portion has a diameter or maximum cross-sectional dimension that is smaller than the diameter or maximum cross-sectional dimension of the port channel, it may be only marginally smaller, such as about 3 mm smaller, about 2 mm smaller, about 1 mm smaller, about 0.5 mm or about 0.1 mm smaller, or otherwise about 10%, about 7%, about 5%, about 3% or about 1% smaller, than the diameter or maximum cross-sectional dimension of the port channel.

The first body portion may be elongate in form and may have a consistent cross-sectional profile along substantially its entire length. Accordingly, depending on its cross-sectional shape, the first body portion may be a square, diamond-shaped, rectangular, hexagonal, or other polygonal prism in shape. However, as discussed above, the first body portion is typically circular in cross-section and hence is substantially cylindrical in shape.

The elongate first body portion may have a longitudinal axis that is longer than the longitudinal axis of the port channel. In such configurations, the first body portion may protrude into the interior of the bioreactor when the sensing probe is operatively engaged with the port. In particular, the first body portion may protrude about 10 mm, about 20 mm, about 30 mm, about 40 mm or about 50 mm into the interior of the bioreactor when the sensing probe is operatively engaged with the port.

The longitudinal axis of the first body portion may have a length from about 50 mm, about 70 mm, about 100 mm, about 150 mm, or about 200 mm to about 250 mm, about 300 mm, about 350 mm, about 400 mm, about 450 mm, about 500 mm, about 600 mm or about 700 mm. The longitudinal axis of the first body portion may also have a length that is greater than about 700 mm.

The second body portion may remain outside the port channel on the exterior of the bioreactor when the probe is operatively engaged with the port. The second body portion may therefore have any suitable size or shape and in particular may have a larger cross-sectional profile than the port channel, and hence also have a larger cross-sectional profile than the first body portion.

The second body portion may also have any cross-sectional shape, and in particular may also be circular, square, diamond-shaped, rectangular, hexagonal, or another polygonal shape in cross-section. However, the cross-sectional shape of the first body portion is typically the same or substantially the same as the cross-sectional shape of the first body portion.

In addition, in embodiments of this invention in which the port with which the probe is intended to be operatively engaged comprises a collar, such as a cylindrical collar, that extending from the wall of the bioreactor that defines the channel of the port, the second body portion may have a similar of the same cross-sectional profile as the exterior surface of the collar.

The second body portion may also be elongate and, depending on its cross-sectional shape may also be cylindrical or a square, diamond-shaped, rectangular, hexagonal, or other polygonal prism in shape.

The elongate first and second body portions may be arranged coaxially. In embodiments in which the cross-sectional profile of the second body portion is larger than the cross-sectional profile of the first body portion, the probe body may further comprise a shoulder located at the interface between the first and second body portions.

In order to facilitate the operative engagement of the sensing probe with the port, the sensing probe may comprise positioning means, which may comprise an over-insertion prevention means. The over-insertion prevention means may comprise one or more projections located on the probe body that prevents that portion of the probe body from entering the port channel. The projections may take any suitable form and in particular may be an annular flange or shoulder, or a series of projections provided on the probe. In some embodiments, the over-insertion prevention means may be the shoulder at the interface between the first body portion and the second body portion.

The second body portion comprises means for enabling the probe to be secured to the port. This means may take any suitable form and in particular may comprise a means for securing the probe to the port or a means for engaging one or more additional components that secure the probe to the port.

Means for securing the probe to the port may comprise one or more engagement members that engage with the port in order to secure the probe to the port. The one or more engagement members may be integrally formed with the second body portion. The one or more engagement members may be configured to engage with one or more projections that are present on the port, such as a cylindrical collar extending from the wall of the bioreactor that defines the channel of the port, or projections such as a barb on the exterior surface of the collar. The one or more engagement members may be formed of a resiliently deformable material and may engage with the port, including projections that are present on the port, by a snap fit or interference fit.

Means for engaging one or more additional components that secure the probe to the port may take any suitable form. In some embodiments, the means for engaging one or more additional components that secure the probe to the port may comprise one or more depressions or projections on the surface of the second body portion. The one or more projections may comprise an annular flange or shoulder around the entire circumference of the second body portion or a series of individual projections provided on the second body portion, which may be regularly spaced around the circumference of the second body portion.

In addition, the one or more additional components that secure the probe to the port may further form a watertight seal between the probe and the port and hence may make the presence of a sealing means on the port unnecessary.

The sensing means is carried on the first body portion of the sensing probe and is exposed to the interior of the bioreactor when the probe is operatively engaged with the port.

The probe may be configured to operatively engage with the port such that the sensing means is at least partly located in the interior of the bioreactor (ie the sensing means is not entirely located in the port channel). In this configuration, the sensing means has greater exposure to the interior of the bioreactor and any biological medium contained within the bioreactor, which may improve the ability of the sensing means to obtain measurements from the biological medium.

In embodiments in which the first body portion has a longitudinal axis that is longer than the longitudinal axis of the port channel such that the first body portion protrudes into the interior of the bioreactor when the sensing probe is operatively engaged with the port, the sensing means may be at least partially located on the region of the first body portion that protrudes into the interior of the bioreactor. Accordingly, the sensing means may be located about 10 mm, about 20 mm, about 30 mm, about 40 mm or about 50 mm into the interior of the bioreactor when the sensing probe is operatively engaged with the port.

The sensing means may take any suitable form depending on the conditions within the bioreactor that the sensing probe is intended to measure. In particular, in embodiments in which the sensing probe is a biomass sensing probe, the sensing means may be at least one electrode. The sensing means of a biomass sensing probe may contain any number of electrodes such as 1, 2, 3, 4, 5, 6, 7, 8 or more than 8 electrodes, or between 2 to 6 or between 2 to 4 electrodes. The at least one electrode may be formed of or plated with gold, stainless steel, iridium, platinum (eg platinum black) or any other material with low or controllable electrode polarisation properties.

The sensing probe may be configured to form a watertight seal with the port when the probe is operatively engaged with the port. A watertight seal may be a seal in which no moisture, or substantially no moisture, is capable of passing through the port, and that is airtight, or substantially airtight, up to 1 bar.

In particular, the sensing probe may comprise one or more sealing members that are configured to form a watertight seal between the body of the probe and the port when the probe is operatively engaged with the port. The sealing members may be configured to form a watertight seal between the interior surface of the port channel and the exterior surface of a region the first body portion that is located within the port channel when the probe is operatively engaged with the port.

In particular, the surface of the first body portion may comprise one or more sealing members that are configured to contact the interior surface of the port channel when the probe is operatively engaged with the port in order to establish a watertight seal. The one or more sealing members may be annular sealing members that extend around the entire circumference of the first body portion. The sealing members may protrude from the surface of the first body portion and may be formed of a resiliently deformable material such as a synthetic rubber including ethylene propylene diene monomer (EPDM) rubber.

Methods of testing the strength of the watertight seal are familiar to the skilled person. In one embodiment, the seal strength may be determined by operatively engaging the probe with a port located on a bioreactor, filling the bioreactor with room temperature water, exerting a pressure of 1 bar within the bioreactor and determining the egress of moisture from the bioreactor via the port. The seal strength formed between the port and the probe preferably permits moisture egress of about 0.1 ml/24 h or less, about 0.05 ml/24 h or less, about 0.02 ml/24 h or less, about 0.01 ml/24 h or less, about 0.005 ml/24 h or less, about 0.002 ml/24 h or less or about 0.001 ml/24 h or less. In some embodiments, no moisture egress via the port may be detected in a 24 hour testing period.

The sensing probe may be for taking any measurements from the interior of a bioreactor. In particular, the sensing probe may be a biomass sensing probe, a pH probe, a temperature probe, a dissolved oxygen probe or a carbon dioxide probe. However, the sensing probe of this invention is preferably a biomass sensing probe.

The sensing probe may be a single-use probe (ie a probe that is intended to be used only once before disposal) or a reusable probe (ie a probe that is intended to be used multiple times before disposal). Single-use probes may be formed of less robust materials, primarily because they do not have to be resistant to repeated sterilisations between uses.

The body of the sensing probe may be formed of any material that meets the functional requirements of the probe. In particular, the body of the probe may be formed of metal or plastics material. The preferred material for forming the body of a single-use sensing probe may be plastic owing to reduced cost and complexity of manufacture of probes made from this material.

Plastics materials that may be used to form the body of the probe include from liquid crystal polymer, phenolic polymer, nylon, polyethylene, polypropylene, polystyrene, polyvinylidene fluoride, polyvinylchloride, acrylonitrile butadiene styrene, acetal resins, sulphone, polysulphone, polyamide, polyphenylene sulphide, polyetheretherketone, polyethylene terephthalate, polyetherketone, polyoxymethylene, polyphthalamide, polyetherketoneketone, thermoplastic polyimide, polyacrylate, polytetrafluoroethylene, polymethyl methacrylate, polycarbonate or mixtures thereof. A currently preferred material for forming the body of the probe is the polycarbonate material commercialised by Bayer under the trade name Makrolon® Rx2530.

The sensing probe must generally be sterile prior to each use and hence may be sterilisable. Accordingly, a single-use sensing probe may be sterilisable once and a sensing probe intended for repeated use may be repeatedly sterilisable. The sensing probe may be sterilisable by gamma irradiation, electron-beam irradiation, heat sterilisation, steam sterilisation or ethylene oxide (ETO) sterilisation.

The body of the sensing probe may be formed of material that is gamma sterilisable or electron-beam sterilisable. In particular, gamma sterilisable materials may be material that does not degrade, deform or soften upon a single exposure or repeated exposures to gamma radiation at a dose of 10 kGy, about 20 kGy, about 25 kGy, about 30 kGy, about 35 kGy, about 40 kGy, about 50 kGy or about 80 kGy, and electron-beam sterilisable materials may be materials that do not degrade, deform or soften upon a single exposure or repeated exposures to electron-beam radiation of about 1 MeV, about 3 MeV, about 5 MeV or about 10 MeV.

The body of the sensing probe may be formed of material that is heat or steam sterilisable. In particular, heat or steam sterilisable materials are materials that have a melting point of greater than about 150° C., greater than about 140° C. greater than about 130° C. or greater than about 120° C.

The probe may be provided pre-sterilised such that it can be used without the need for sterilisation by the user. The pre-sterilised probe may be provided in sterile packaging.

The body of the sensing probe may be monolithic (ie formed from a single component) or modular (ie formed from a plurality of probe body components).

The assembly of sensing probes with adhesive may be problematic in certain applications as compounds within the adhesive can promote reactions within a bioreactor or otherwise contaminate or have other deleterious effects on biomass contained within the bioreactor. Additionally, any adhesive that is used to produce the biomass sensing probe may become separated from the probe and travel into the biological medium, which can exacerbate contamination or other adverse effects upon the medium.

Accordingly, any adhesive that is used in the body of the probe, or any portion of the probe that is exposed to the interior of the bioreactor when the probe is operatively engaged with a port, are preferably medically approved and biocompatible adhesives, such as adhesives that are adhesives are biocompatible under ISO 10993 and USP class VI. Alternatively, the body of the probe, or any portion of the probe that is exposed to the interior of the bioreactor when the probe is operatively engaged with a port, may be free of adhesive. In particular, the body of the probe, or any portion of the probe that is exposed to the interior of the bioreactor when the probe is operatively engaged with a port, may either be formed from a single component or formed from a plurality of probe body components that are secured together without the use of adhesives.

In arrangements in which the body of the probe or the portion of the probe that is exposed to the interior of the bioreactor when the probe is operatively engaged with a port comprises a plurality of probe body components, those separate body components may be secured together without the use of adhesive using solvents (eg methylene chloride, ethylene dichloride, acetone, or a mixture thereof), mechanical connection (eg snap fit, friction fit or the like), by heat welding or by ultrasonic welding. Appropriate adhesive-free securing method will depend at least on the materials from which the probe body components are formed and will be apparent to the skilled person.

In a modular arrangement, the body of the sensing probe may comprise a core, which may provide the probe with sufficient rigidity to enable it to be operatively engaged with the port, and a shell, which may provide the body of the probe with the desired external profile for operatively engaging with a port. In such embodiments, the shell may be monolithic or may be modular. The shell may enclose the core entirely or may partially enclose the core such that a portion of the core is exposed. The portion of the core that is exposed may be located on the portion of the probe that is exposed to the interior of the bioreactor when the probe is in an operative position.

The body of the sensing probe, or any portion of the probe that is exposed to the interior of the bioreactor when the probe is operatively engaged with a port, may be watertight. The probe may be watertight if no moisture, or substantially no moisture, is capable of entering the probe from its exterior. In particular, leakage of moisture, such as the biological medium housed within a bioreactor, into the interior or the probe may have undesirable effects on the functioning of the probe, may result in loss of the biological medium and may also risk contaminating the biological medium potentially resulting in spoilage of the whole batch.

Methods of testing whether the probe body or at least the portion of the probe that is exposed to the interior of the bioreactor when the probe is operatively engaged with the port is watertight are familiar to the skilled person. In particular, a determination of whether or not the probe is watertight can be made by submerging the probe, or the portion of the probe that is exposed to the interior of the bioreactor when the probe is in an operative position in water in a vessel at a temperature of 25° C. and a pressure of 1 bar for thirty minutes, although time and pressure may vary. Water ingress into the probe can be determined by weight gain, in which the mass of the probe before and after submersion may be taken, with a weight gain of less than about 0.1 g, less than about 0.05 g, less than about 0.02 g, less than about 0.01 g or 0 g indicates that no moisture has entered the probe.

The probe may further comprise a communication means for communicating the signal from the sensing means to a signal processing means, such as a biomass signal processing means. Those skilled in the art will be familiar with biomass signal processing means, for example the Futura® range of systems (Aber Instruments Limited).

The communication means may comprise means for communicating with a signal processing means via a cable, for example such that the cable may be removably coupled to the probe. The communication means may comprise a plug/socket, a slide connector, a push-pull connector, such as the connector commercialised by Redel®, a flexible catch, a magnetic connector or a screw connector. Alternatively, the communication means may be a wireless communication means, such as a means for communication with a signal processing means via WLAN, bluetooth, RFID, NFC, or the like.

The probe may further comprise a signal transmission means for transmitting the signal from the sensing means to a location remote from the sensing means, such as the communication means. The signal transmission means may comprise tracks, which may be formed on a printed circuit board (PCB), wires, fibre optic cables, or any other suitable means. The body of the sensing probe may be hollow to permit signal transmission means to pass through its interior of the probe. Alternatively, the probe body may be solid and may optionally have the signal transmission means moulded into its interior.

The probe may comprise data storage means, such as a microchip. The data storage means may store information such as calibration correction values, serial numbers, part numbers, temperature values or other information.

According to a second aspect of this invention, there is provided an assembly comprising a sensing probe according to the first aspect of this invention and a port comprising a channel that communicates between the interior and the exterior of a bioreactor, where in the probe is operatively engaged with the port.

The probe and port according to this aspect of the invention may comprise any combination of the features discussed above in relation to the first aspect of this invention.

As the probe is operatively engaged with the port, the first body portion of the probe is at least partially located within the port channel and the sensing means of the probe is exposed to the interior of the bioreactor and may be at least partly located in the interior of the bioreactor (ie the sensing means is not entirely located in the port channel).

The probe may comprise positioning means, which may comprise an over-insertion prevention means. The over-insertion prevention means may comprise one or more projections located on the probe body, such as an annular flange or shoulder, and may comprise the shoulder at the interface between the first body portion and the second body portion. Accordingly, in the assembly according to this aspect of the invention, the over-insertion prevention means may be abutted against the port, such as the rim of the opening at the exterior end or the port channel.

The port may comprise a collar that projects from the exterior of the wall and that defines the channel of the port and may further comprise one or more projections on the exterior surface of the collar. The one or more projections may be annular projections and may extend around the entire circumference of the exterior wall of the collar, and in particular may comprise one or more barbs that comprise a shoulder on the side of the barb that is distal from the exterior end of the collar.

The second body portion of the probe comprises means for enabling the probe to be secured to the port and the probe may be secured to the port by such means in the assembly according to this aspect of the invention.

The means for enabling the probe to be secured to the port may comprise means for securing the probe to the port, which may comprise one or more engagement members that engage with the port in order to secure the probe to the port. In particular, the one or more engagement members may be formed of a resiliently deformable material and may engage with the port by a snap fit or interference fit. The one or more engagement members may engage with projections on the port and in particular may engage with a barb shoulder located on the exterior of the port collar.

The means for enabling the probe to be secured to the port may comprise means for engaging one or more additional components that secure the probe to the port, in which case the assembly may further comprise such one or more additional components. In some embodiments, the means for engaging one or more additional components that secure the probe to the port may comprise one or more depressions or projections on the surface of the second body portion. The one or more projections may comprise an annular flange or shoulder around the entire circumference of the second body portion or a series of individual projections provided on the second body portion, which may be regularly spaced around the circumference of the second body portion. In some embodiments, the one or more projections may comprise a barb that comprises a shoulder on the side of the barb that is distal from the first body portion of the probe.

The one or more additional components may be any suitable means capable of engaging the second body portion and the port in order to secure the probe to the port. In particular, the additional components may engage one or more depressions or projections on the second body portion, projections on the port collar, or both, in order to secure the probe to the port.

The one or more additional components may comprise a sheath. The sheath may surround the assembly such that at least a portion of each of the second body portion and the collar of the port are surrounded. The internal surface of the sheath may engage with depressions or projections on the second body portion, projections on the collar of the port, or both.

The second body portion may have a similar or the same cross-sectional profile as the exterior surface of the collar, in which case the sheath may be of substantially consistent cross-sectional profile.

The sheath may comprise any suitable material and in particular may comprise a waterproof material such that the sheath forms a watertight seal over the assembly.

The sheath may be formed of resilient material and may fit over the second body portion and the collar of the port with an interference fit. In particular, the sheath may be a length of tube of the appropriate diameter for engaging the bioreactor port. The sheath may be cut to the appropriate length before being placed over the assembly.

The sheath may be secured in place in the assembly by securing means. The securing means may secure the sheath in the portion overlying the second body portion of the probe and in the portion overlying the collar of the port. In particular, the securing means may secure the sheath to the second body portion in a region overlying or adjacent to the one or more depressions or projections present on the second body portion, or more specifically may secure the sheath to the second body portion on the side of the one or more depressions or projections that is distal from the first body portion.

In particular, the securing means may secure the sheath to the port collar in a region overlying or adjacent to the one or more projections present on the port collar, or more specifically may secure the sheath to the port collar portion on the side of the one or more port collar projections that is distal from the opening at the exterior end or the port channel.

The securing means may be of the type that is conventionally used to secure tubes to bioreactor ports and may include cable ties, hose clamps or the like.

Alternatively, the one or more additional components that may be engaged with the means for enabling the probe to be secured to the port may comprise a probe retention clip. The probe retention clip may comprise a base that may engage one or more depressions or projections on the second body portion of the probe. The clip may further comprise one or more engagement members that engage with the port in order to secure the probe to the port. The one or more engagement members may be formed of a resiliently deformable material and may engage with the port by a snap fit or interference fit. In particular, the one or more engagement members may engage with projections on the port and in particular may engage with a barb shoulder on the port.

According to a third aspect of this invention, there is provided a kit comprising a probe according to the first aspect of this invention and one or more additional components for securing the probe to a port comprising a channel that communicates between the interior and exterior of a bioreactor.

The one or more additional components may comprise a sheath, sheath securing means, probe retention clip, or any combination thereof.

According to a fourth aspect of this invention, there is provided a system comprising a bioreactor having a port that comprises a channel that communicates between the interior and the exterior of the bioreactor, and a sensing probe according to the first aspect of this invention that is operatively engaged with the port in order to provide an assembly.

The probe, port and assembly according to this aspect of the invention may comprise any combination of the features discussed above in relation to the other aspects of this invention.

The bioreactor may be a single-use bioreactor (ie a bioreactor that is intended to be used only once before disposal), or may be a reusable bioreactor (ie a bioreactor that is intended to be used multiple times before disposal).

Single-use bioreactors include “bag-type” bioreactors such as those marketed by Applikon, Broadley James, Cellexus, Eppendorf, Finesse, GE Lifesciences, Infors, Pall or Sartorius Stedim, or “vessel-type” bioreactors such as the bioreactor marketed under the brand name HyPerforma® by Thermo-Fisher.

Reusable bioreactors may be fermenters formed from stainless steel, glass or plastic, and may have a capacity ranging from sub-litre, such as 10 ml to about 950 ml, to industrial scale such as about 10,000 litres, about 50,000 litres or about 100,000 litres.

The bioreactor may be for use in the brewing industry or the biotechnology industry. The bioreactor may house, or be for use in housing, any biological medium. The biological medium may be a liquid containing a plurality of cells of one or more types or sub-types, which may be human cells, non-human animal cells (including mammalian and non-mammalian cells), bacteria, plant cells, fungal cells (including yeast cells), stem cells, cells of an immortal or immortalised cell line, hybridoma cells, or any other cells.

According to a fifth aspect of this invention, there is provided a method of obtaining a measurement from the interior of a bioreactor, the method comprising the steps of operatively engaging a probe according to the first aspect of this invention with a port that comprises a channel that communicates between the interior and the exterior of a bioreactor in order to provide an assembly and obtaining one or more measurements from the interior of the bioreactor.

The probe, port, assembly and bioreactor according to this aspect of the invention may comprise any combination of the features discussed above in relation to the other aspects of this invention.

The method may further comprise sterilising the probe prior to its operative engagement with the port.

In addition, the method may comprise calibrating the probe following its operative engagement with the port.

Specific embodiments of the invention are described in further detail below by way of example only with reference to the accompanying drawings, in which:

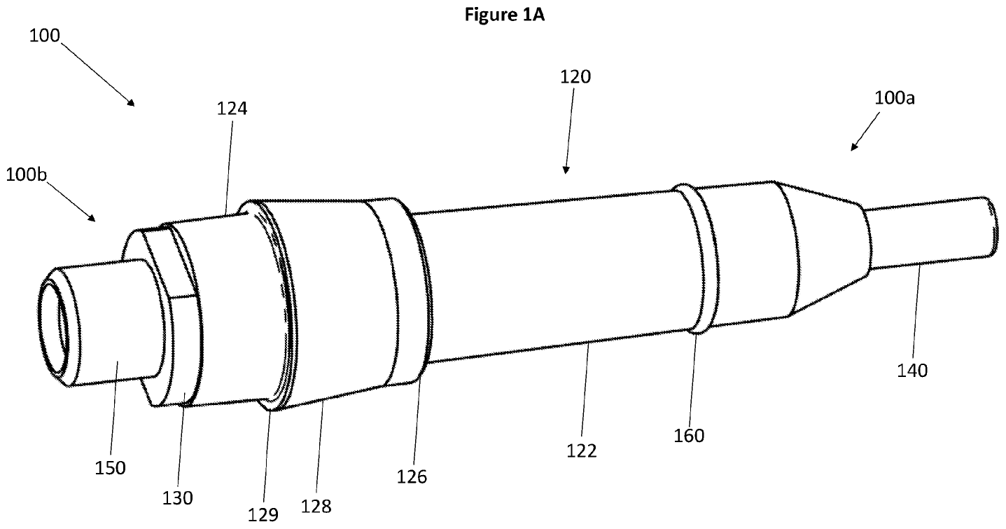

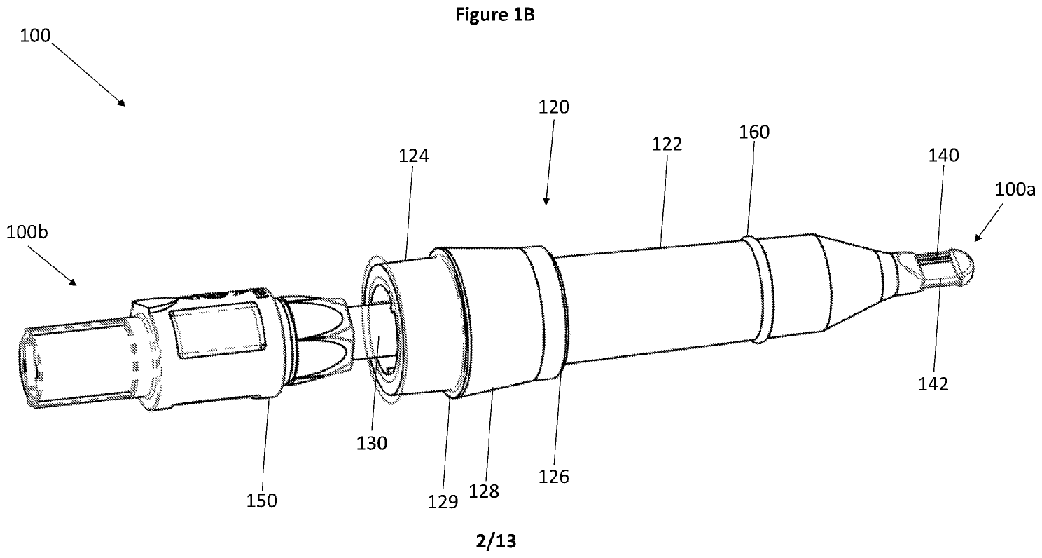

FIG. 1A is a perspective view of a generic sensing probe according to the invention;

FIG. 1B is a perspective view of a biomass sensing probe according to the invention;

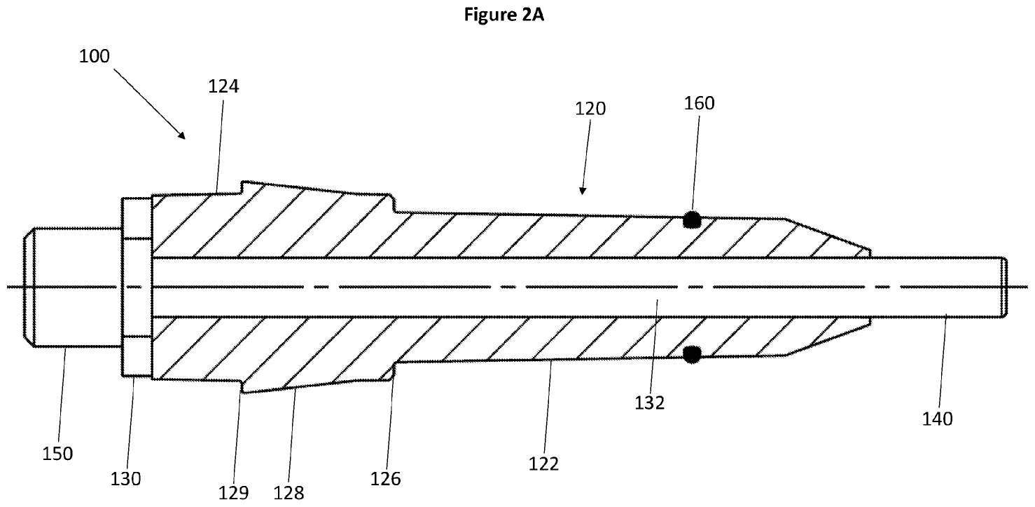

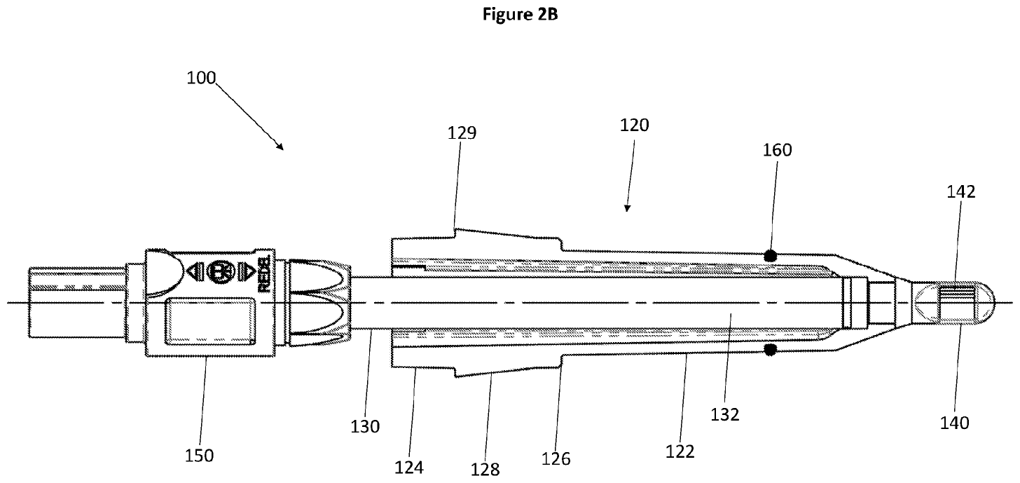

FIG. 2A is a longitudinal cross-sectional view of the generic sensing probe of FIG. 1A;

FIG. 2B is a longitudinal cross-sectional view of the biomass sensing probe of FIG. 1B;

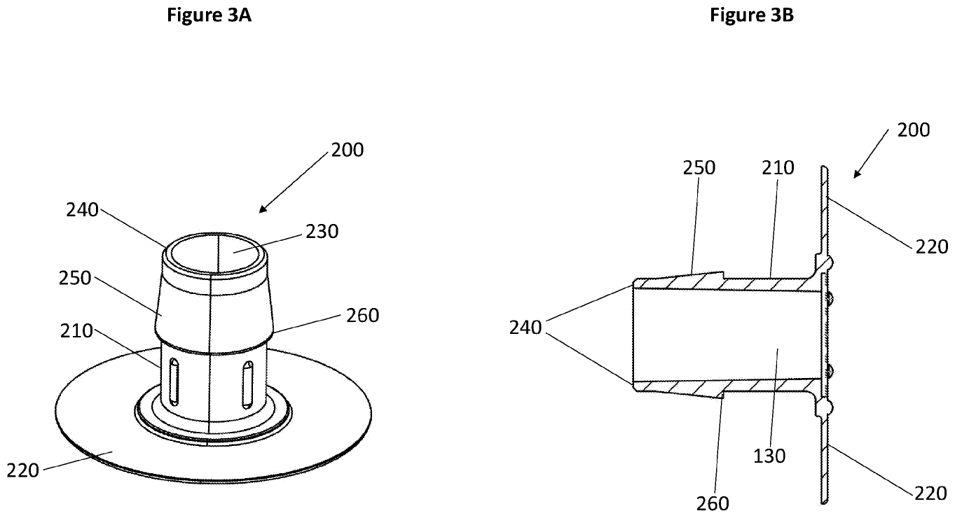

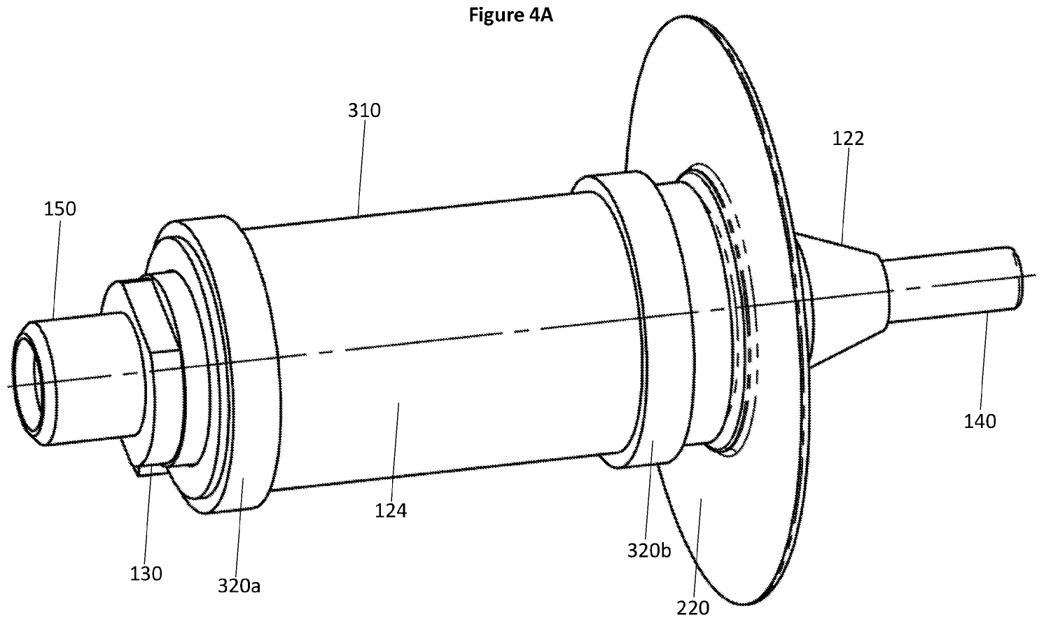

FIG. 3A is a perspective view of a ¾″ Eldon James port that may be engaged by the sensing probe of FIG. 1A or 1B;

FIG. 3B is a cross-sectional view of the ¾″ Eldon James port illustrated in FIG. 3A;

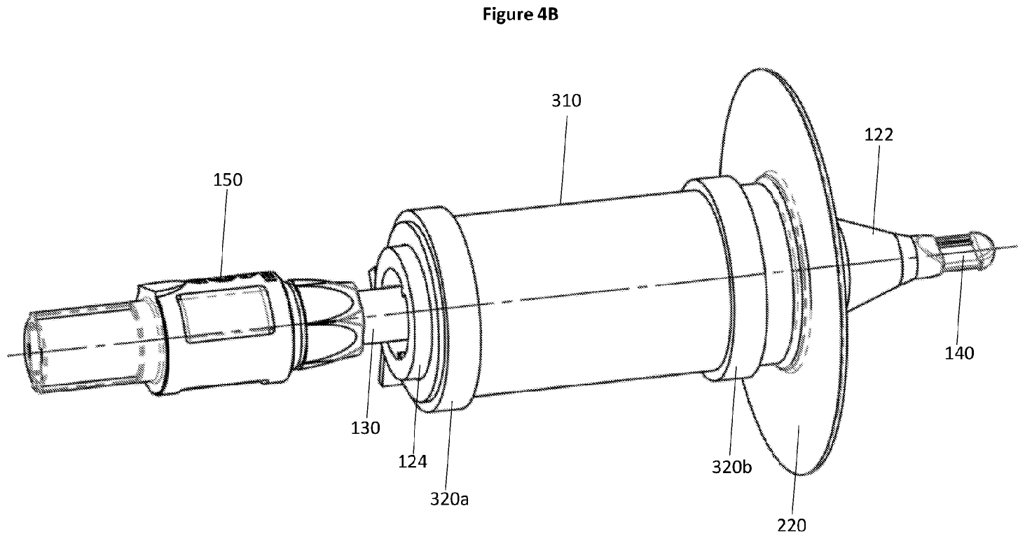

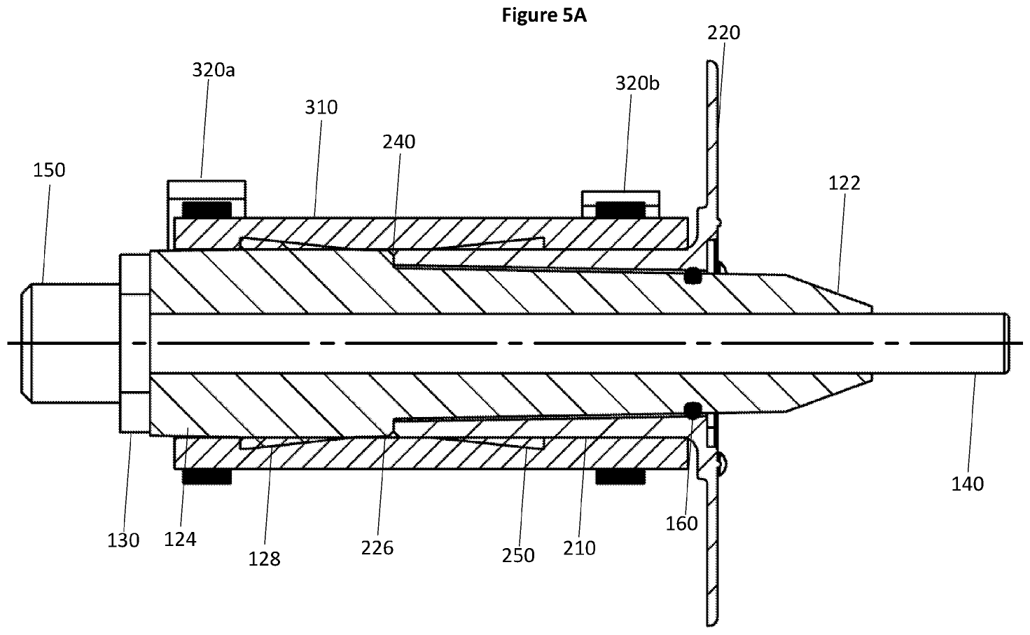

FIG. 4A is a perspective view of the generic sensing probe of FIG. 1A operatively engaged with the port of FIGS. 3A and 3B according to a first arrangement;

FIG. 4B is a perspective view of the biomass sensing probe of FIG. 1B operatively engaged with the port of FIGS. 3A and 3B according to a first arrangement;

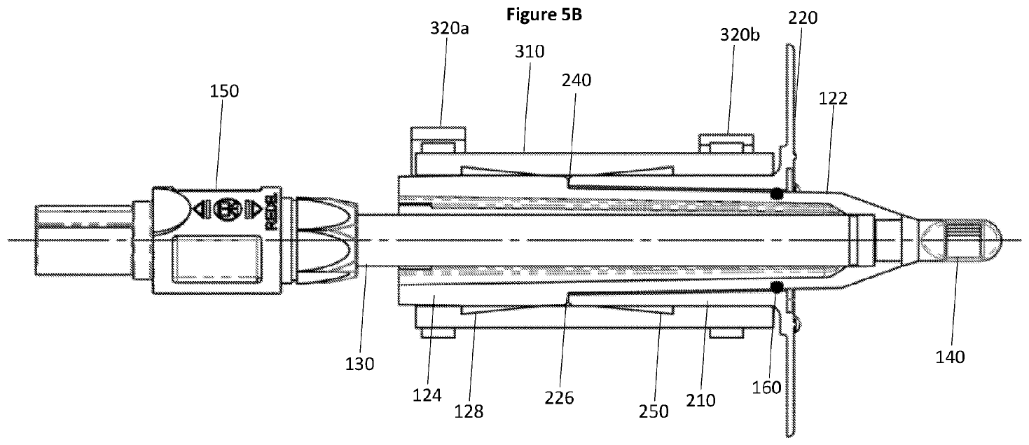

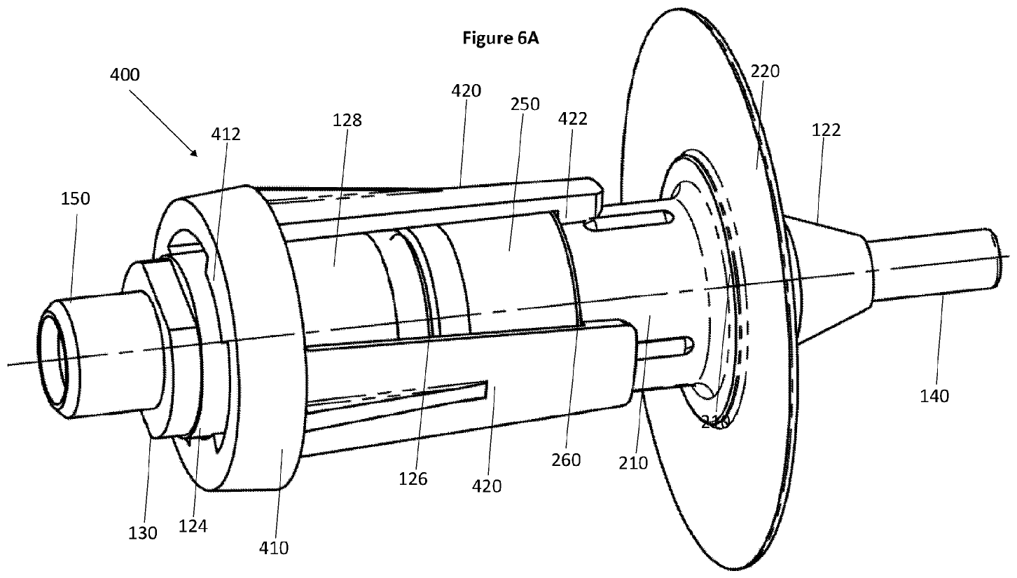

FIG. 5A is a longitudinal cross-sectional view of the arrangement of FIG. 4A;

FIG. 5B is a longitudinal cross-sectional view of the arrangement of FIG. 4B;

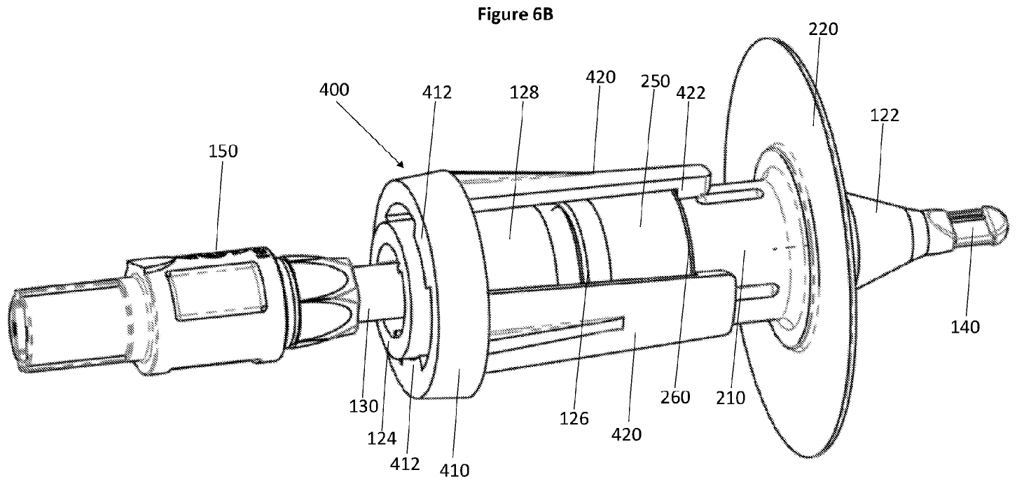

FIG. 6A is a perspective view of the generic sensing probe of FIG. 1A operatively engaged with the port of FIGS. 3A and 3B according to a second arrangement;

FIG. 6B is a perspective view of the biomass sensing probe of FIG. 1B operatively engaged with the port of FIGS. 3A and 3B according to a second arrangement;

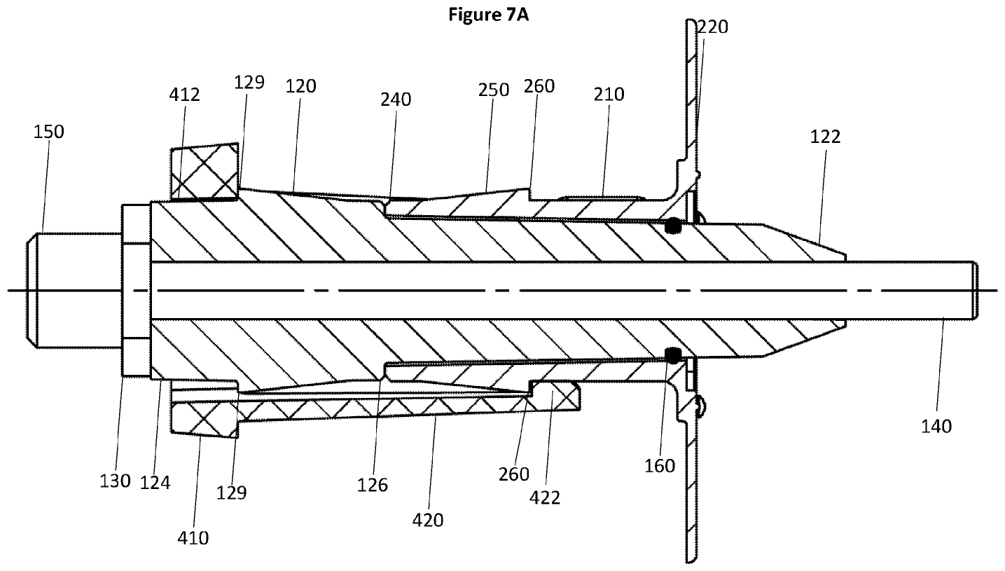

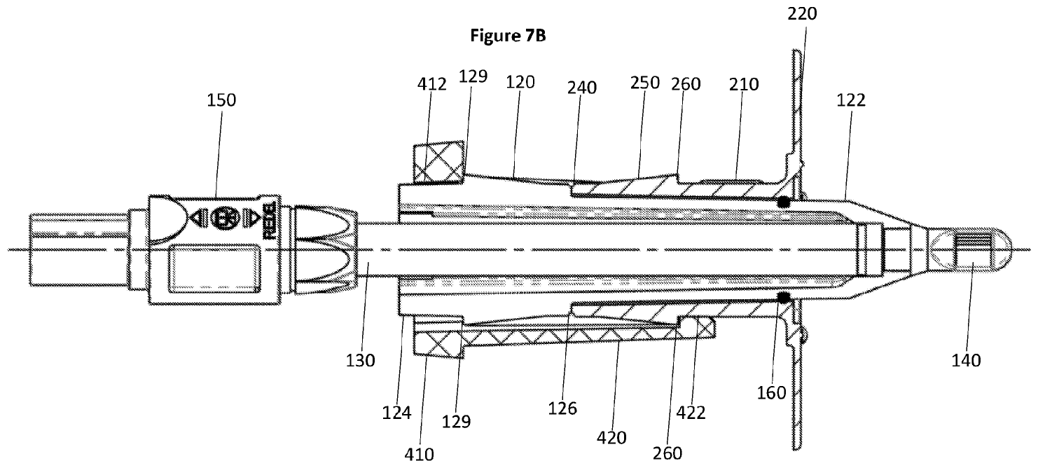

FIG. 7A is a longitudinal cross-sectional view of the arrangement of FIG. 6A; and,

FIG. 7B is a longitudinal cross-sectional view of the arrangement of FIG. 6B.

FIGS. 1A and 2A illustrate a generic sensing probe 100 according to this invention, which may be for sensing any of the conditions inside a bioreactor or any similar vessel and in particular may be a biomass sensing probe, a pH probe, a temperature probe, a dissolved oxygen probe or a carbon dioxide probe, while FIGS. 1B and 2B illustrate a biomass sensing probe 110 according to this invention. The probe 100, 110 is elongate in shape, having a distal end 100a, 110a and a proximal end 100b, 110b, and comprises an outer shell 120 that surrounds an inner core 130.

The distal end of the core 130 protrudes from the distal end of the shell 120 and bears a sensing tip 140 that, in the case of the biomass sensing probe, comprises four electrodes 142, two of which are visible in FIGS. 1B and 2B. The proximal end of the core 130 also protrudes from the proximal end of the shell 120 and is provided with coupling means 150 which, in the case of the biomass sensing probe, is in the form of a Redel push-pull connector, but in any case may be any connector that is suitable for connecting the probe 100, 110 to a biomass signal processing means (not shown). The portion of the core 130 contained within the shell 120 provides a space 132 through which the signal from the sensing tip 140 is transmitted to the coupling means 150 via a signal transmission means (not shown).

The shell 120 comprises a first portion 122 at its distal end, a second portion 124 at its proximal end and a shoulder 126 located at the interface between the first 122 and second 124 portions. The first and second portions 122, 124 are generally cylindrical in shape and the first portion 122 having a narrower diameter than the second portion 124. The first portion 122 comprises an annular sealing member 160 formed of silicone, which marginally protrudes from the surface of the shell 120 around its entire circumference. The distal end of the first portion 122 comprises a tapered region that forms a point at the distal end of the shell 120 from which the distal end of the core 130, which bears the sensing tip 140, protrudes. The second portion 124 of the shell comprises an annular barb 128 that protrudes from the surface of the shell 120 around its entire circumference and provides a barb shoulder 129 on its proximal side.

The shell 120, core 130 and sensing tip 140 are formed of polycarbonate material commercialised by Bayer under the trade name Makrolon® Rx2530 and are sealed together with the use of a UV curable adhesive that is biocompatible under ISO 10993 and USP class VI available from Dymax Corporation.

FIGS. 3A and 3B illustrate a ¾″ Eldon James port 200, which is a standard type of port that is conventionally present in the wall of a bioreactor. The port 200 comprises a cylindrical collar 210 and an annular flange 220 extending outwardly from one end of the collar 210. The cylindrical collar 210 is open at both ends and defines a channel 230 of a constant circular cross-section.

The collar 210 at the end opposite to the flange 220 comprises a rim 240. The exterior surface of the collar 210 adjacent to the rim 240 is flared in order to form a barb 250 that protrudes from the exterior surface of the collar 210 around its entire circumference and provides a barb shoulder 260 on the side opposite the rim 240.

The surface of the flange 220 that is opposite to the collar 210 will generally be fastened to the exterior wall of a bioreactor (not shown), such as by heat or ultrasonic welding, such that the collar 210 extends outwardly from the exterior of the bioreactor. In addition, the port 200 is fastened to the exterior wall of the bioreactor such that the opening at the end of the collar 210 that is adjacent to the flange 220 is in registration with an opening in the wall of the bioreactor such that the channel 230 communicates between the interior and the exterior of the bioreactor.

FIGS. 4A, 5A, 6A and 7A illustrate assemblies in which the generic probe 100 of FIGS. 1A and 2A is operatively engaged with the port 200 of FIGS. 3A and 3B and the probe 100 and port 200 are fastened together according to a first arrangement (FIGS. 4A and 5A) or a second arrangement (FIGS. 6A and 7A). FIGS. 4B, 5B, 6B and 7B illustrate assemblies in which the biomass sensing probe 110 of FIGS. 1B and 2B is operatively engaged with the port 200 of FIGS. 3A and 3B and the probe 110 and port 200 are fastened together according to a first arrangement (FIGS. 4B and 5B) or a second arrangement (FIGS. 6B and 7B).

The diameter of the first portion of the probe shell 122 is marginally smaller than and the internal diameter of the port channel 230. The first portion of the probe shell 122 can therefore be freely inserted into the channel 230 until the shoulder 126 abuts the rim of the collar 240, at which point the probe 100, 110 is located in its operative position within the port 200.

The external diameter of the second portion of the probe shell 124 is roughly the same as the external diameter of the port collar 210, and the barbs located on the second portion of the probe shell and the port collar 128, 250 have roughly the same profile. Accordingly, when the probe 100, 110 is located in the operative position within the port 200, the exterior of the assembly is a cylinder of roughly constant diameter that is made up of the second portion of the probe shell 124 and the port collar 210, and which carries two opposing barbs 128, 250.

The length of the first portion of the probe shell 122 is slightly greater than the length of the channel 230 such that, when the probe 100, 110 is located in the operative position, the sensing tip 140 projects from the opening at the opposite end of the channel 230 and hence into the interior of the bioreactor (not shown).

The annular seal 160 is located within the channel 230 when the probe is in the operative position (see FIGS. 5A, 5B, 7A and 7B). The annular seal 160 marginally protrudes from the surface of the probe shell 120 and makes contact with the internal surface of the channel 230 around its entire circumference in order to create a watertight seal that prevents any liquid contained within the bioreactor from escaping via the port 200.

Referring now to FIGS. 4A, 4B, 5A and 5B, the probe may be fastened in its operative position by a first means in which a sleeve 310 is placed over the probe 100, 110 and port 200 assembly. The sleeve 310 is a piece of standard polycarbonate tube having an internal diameter that is the same as the external diameter of the probe shell 124 and the port collar 210, and which has been cut to the appropriate length.

A first fastener 320a is then fastened around the sleeve 310 at a position that overlays the second portion 124 of the probe shell on the proximal side of the barb 128 and a second fastener 320b is fastened around the sleeve 310 at a position that overlays the collar 210 of the port on the distal side of the port barb 250. The probe 100, 110 is thereby firmly held in its operative position by the sleeve 310 and fasteners 320a, 320b.

This first means of fastening the probe 100, 110 in its operative position also forms a watertight seal at both ends of the sleeve 310 around the second portion 124 of the probe shell and around the base of the collar 210 of the port. Accordingly, when this means of fastening the probe in its operative position is used, it is to be understood that the annular seal 160 on the first portion of the probe shell 122 is not required in order to form a watertight seal between the probe 100, 110 and the port 200.

In addition, although in this exemplary embodiment the probe is engaged with the port depicted in the FIGS. 3A and 3B, this first means of fastening the probe 100, 110 in its operative position is also suitable for use in arrangements comprising a wide range of port designs, and in particular it is not necessary for the port to comprise a barb having the same profile as the barb located on the second portion of the shell 124.

Referring now to FIGS. 6A, 6B, 7A and 7B, the probe may be fastened in its operative position by a second means in which a clip 400 is placed over the probe 100, 110 and port 200 assembly.

The clip is formed of Makrolon® polycarbonate and comprises an annular base 410 from which three equally spaced arms 420 extend. The annular base 410 comprises a central opening having a diameter that is greater than the external diameter of the second portion 124 of the probe shell. The base 410 further comprises projections 412 that project into the opening such that the minimum diameter of the opening is less than the diameter of the barb 128. The arms 420 each have a length that is equal to the spacing between the barb shoulder 129 on the second portion 124 of the probe shell and the barb shoulder 260 located in the collar 210 of the port when the probe is in its operative position. The arms 420 each comprise a hook 422 at their distal end.

The clip 400 is placed over probe 100, 110 and port 200 assembly such that the opening in the base of the clip 410 is placed over second portion 124 of the probe shell until the projections 412 abut the barb shoulder 129. In this position, the arms 420 of the clip extend over the assembly towards the flange 220 of the port and their hooks 422 engage with the shoulder 260 of the barb located on the port collar with a snap fit. The probe 100, 110 is thereby firmly held in its operative position by the engagement of the projections 412 with the barb shoulder 129 located on the second portion 124 of the probe shell and the engagement of the hooks 422 with the barb shoulder 260 located on the collar 210 of the port.

1. A sensing probe for operatively engaging with a port that comprises a channel that communicates between the interior and the exterior of a bioreactor, the probe comprising;
an elongate body comprising;
a first body portion that is locatable within the port channel; and,
a second body portion comprising means for enabling the probe to be secured to the port; and,
a sensing means located on the first body portion,
wherein the probe is configured to operatively engage with the port such that the first body portion is at least partially located within the port channel and the sensing means is exposed to the interior of the bioreactor.

2. The probe according to claim 1, wherein the first body portion has a cross-sectional profile that is the same as or marginally smaller than the cross-sectional profile of the port channel.

3. The probe according to claim 2, wherein the diameter or maximum cross-sectional dimension of the port channel is about 3 mm smaller, about 2 mm smaller, about 1 mm smaller, about 0.5 mm or about 0.1 mm smaller, or otherwise about 10%, about 7%, about 5%, about 3% or about 1% smaller, than the diameter or maximum cross-sectional dimension of the port channel.

4. The probe according to any preceding claim, wherein the first body portion is substantially cylindrical in shape.

5. The probe according to any preceding claim, wherein the first body portion protrudes into the interior of the bioreactor, optionally by about 10 mm, about 20 mm, about 30 mm, about 40 mm or about 50 mm, when operatively engaged with the port.

6. The probe according to any preceding claim, wherein the second body portion is located outside the port channel on the exterior of the bioreactor when the probe is operatively engaged with the port.

7. The probe according to any preceding claim, wherein the second body portion has a larger cross-sectional profile than the first body portion.

8. The probe according to any preceding claim, wherein the first and second body portions are elongate and arranged coaxially.

9. The probe according to any preceding claim, wherein the probe body further comprises a shoulder located at the interface between the first and second body portions.

10. The probe according to claim 9, wherein the shoulder is configured to abut the port when the probe is operatively engaged with the port.

11. The probe according to any preceding claim, wherein the means for enabling the probe to be secured to the port comprises means for securing the probe to the port or means for engaging one or more additional components that secure the probe to the port.

12. The probe according to claim 11, wherein the means for engaging one or more additional components comprise one or more depressions or projections located on the second body portion.

13. The probe according to any preceding claim, wherein the probe is configured to operatively engage with the port such that the sensing means is at least partly located in the interior of the bioreactor and optionally about 10 mm, about 20 mm, about 30 mm, about 40 mm or about 50 mm into the interior of the bioreactor, when the sensing probe is operatively engaged with the port.

14. The probe according to any preceding claim, wherein the probe is configured to form a watertight seal with the port when the probe is operatively engaged with the port.

15. The probe according to any preceding claim, wherein the first body portion comprises one or more sealing members that are configured to contact the interior surface of the port channel when the probe is operatively engaged with the port.

16. The probe according to any preceding claim, which is a biomass sensing probe, a pH probe, a temperature probe, a dissolved oxygen probe or a carbon dioxide probe.

17. The probe according to any preceding claim, which is a single-use probe.

18. The probe according to any preceding claim, wherein the probe body is formed of material that is sterilisable by gamma irradiation, electron-beam irradiation, heat sterilisation, steam sterilisation or ethylene oxide (ETO) sterilisation.

19. The probe according to any preceding claim, the probe body is formed of a single component, or is formed of a plurality of components that are fastened together with the use of medically approved and biocompatible adhesives or without the use of an adhesive.

20. An assembly comprising a probe according to any of claims 1 to 19 and a port comprising a channel that communicates between the interior and the exterior of a bioreactor, wherein the probe is operatively engaged with the port.

21. The assembly of claim 20, wherein the probe comprises an over-insertion prevention means comprising one or more projections abutted against the port.

22. The assembly of any of claims 20 or 21 further comprising additional components engaged with the second body portion and the port in order to secure the probe to the port, optionally wherein the additional components comprise
a sheath that surrounds the assembly, and optionally one or more securing means for securing the sheath to the second body portion and the port; or,
a probe retention clip engaged with the second body portion and the port.

23. A kit comprising a probe according to any of claims 1 to 19 and one or more additional components for securing the probe to a port comprising a channel that communicates between the interior and exterior of a bioreactor.

24. A system comprising a bioreactor having a port that comprises a channel that communicates between the interior and the exterior of the bioreactor, and a probe according to any of claims 1 to 19 that is operatively engaged with the port in order to provide an assembly.

25. A method of obtaining a measurement from the interior of a bioreactor, the method comprising the steps of operatively engaging a probe according to any of claims 1 to 19 with a port that comprises a channel that communicates between the interior and the exterior of a bioreactor in order to provide an assembly and obtaining one or more measurements from the interior of the bioreactor.
